SEQUENCE LISTING (6 sequences)

SEQ ID NO: 1 - DNA, 1773 bp, Escherichia coli
  misc_feature (1)..(1773): Putative outer-membrane transporter essential for contact-dependent inhibition
gagaatatgcggcaccggcaggataacttactggcaaacagaacattgttgcccggtatggcttccggtcagtacgtattcaggctctgcactttctctccggtggtacgctatttttccctcctcccctgcctttgtattctttcgttttcgtctccggcagccatgctgtctccgggtgaccgcagtgcaattcagcagcaacagcagcagttgctggatgaaaaccagcgtcagcgtgatgcgctggagcgcagtgcgccgctgaccatcacgccgtctccggaaacgtctgccggtactgaaggtccctgctttacggtgtcaagcattgttgtcagtggggccacccgactgacgtctgcagaaaccgacagactggtggcaccgtgggtgaatcagtgtctgaatatcacggggctgaccgcggtcacggatgccatgacggacagctatatacgccggggatatatcaccagccgggcctttctgacagagcaggacctttcagggggcgtactgcacataacggtcatggaaggcaggctgcagcaaatccgggcggaaggcgctgaccttcctgcccgcaccctgaagatggttttcccgggaatggaggggaaggttctgaacctgcgggatattgagcaggggatggagcagattaatcgtctgcgtacggagccggtacagattgaaatatcgcccggtgaccgtgagggatggtcggtggtgacactgacggcattgccggaatggcctgtcacagggagcgtgggcatcgacaacagcgggcagaagagtaccggtacggggcagttaaatggtgtcctttcctttaataatcctctggggctggctgacaactggtttgtcagcgggggacggagcagtgacttttcggtgtcacatgatgcgaggaattttgccgccggtgtcagtctgccgtatggctataccctggtggattacacgtattcatggagtgactacctcagcaccattgataaccggggctggcggtggcgttccacgggagacctgcagactcaccggctgggactgtcgcatgtcctgttccgtaacggggacatgaagacagcactgaccggaggtctgcagcaccgcattattcacaattatctggatgatgttctgcttcagggcagcagccgtaaactcacttcattttctgtcgggctgaatcacacccacaagtttctggggggggtcggaacactgaatccggtattcacacgggggatgccctggttcggcgcagaaagcgaccacgggaaaaggggagacctgcccgtaaatcagttccggaaatggtcggtgagtgccagttttcagcgccccgtcacggacagggtgtggtggctgaccagcgcttatgcccagtggtcaccggaccgtcttcatggtgtggaacaactgagcctcgggggtgagagttcagtgcgtggctttaaggatcagtatatctccggtaataacggcggttatctgcggaatgagctgtcctggtctctgttctccctgccatatgtgggaactgtccgtgcagtggctgcactggacggcggctggctgcactctgacagcgatgacccgtactcgtccggcacgctgtggggtgctgctgccgggctcagcaccaccagtggccatgtttccggttcgttcactgccggactgcctctggtttacccggactggcttgcccctgaccatctcacggtttactggcgcgttgccgtcgcgttttaa

SEQ ID NO: 2 - DNA, 9399 bp, Escherichia coli
  misc_feature (1)..(9399): Secreted cell-surface protein essential for contact-dependentinhibition
atgcatcagcctcccgttcgcttcacttaccgcctgctgagttacctggtcagtgcgattatcgccgggcagccgttgttaccggctgtgggggccgtcatcaccccacaaaacggggccggaatggataaagcggcaaatggtgtgccggtcgtgaacattgccacgccgaacggggccgggatttcgcataaccggtttacggattacaacgtcgggaaggaagggctgattctcaataatgccaccggtaagcttaatccgacgcagcttggtggactgatacagaataacccgaacctgaaagcgggcggggaagcgaagggtatcatcaacgaagtgaccggcggtaagcgttcactgctgcagggctatacggaagtggccggcaaagcggcgaatgtgatggttgccaacccgtatggtatcacctgtgacggctgtggctttatcaacacgccgcacgcgacgctcaccacgggcaaacctgtgatgaatgccgacggcagcctgcaggcgctggaggtgactgaaggcagtatcaccatcaatggcgcgggcctggacggcacccggagcgatgccgtatccattattgcccgtgcaacggaagtgaatgccgcgcttcatgcgaaggatttaactgtcactgcaggcgctaaccggataactgcagatggtcgcgtcagtgccctgaagggcgaaggtaatgtgccgaaagttgccgttgataccggcgctctcggtggaatgtacgccaggcgtattcatctgacctccactgaaagtggtgtcggggttaatcttggtaacctttatgcccgcgagggcgatatcatactgagcagttccggaaaactggtcctgaagaacagccttgccggcggcaataccaccgtaaccggaacggatgtctcactttcaggggataacaaagccggaggaaatctcagcgttaccgggacaacgggactgacactgaatcagtcccgtctggtgacggataaaaatctggtgctgtcttcatccgggcagattgtacagaacggtggtgaactgactgccgggcagaacgccatgctcagtgcacagcacctgaaccagacttccgggaccgtgaatgcagctgaaaatgtcaccttgaccaccaccgatgataccacactgaaaggccgcagcgttgccgggaaaacactcaccgtcagttccggcagcctgaacaacggtgggacactggttgccgggcgcgatgccacggtgaaaaccgggacattcagtaataccggtaccgtccaggggaatggcctgaaagttaccgccactgacctgaccagcaccggcagtattaaaagtggcagcacactcgatatcagcgcccgcaatgccacactgtccggtgatgccggagcaaaagacagagccctcgttaccgtcagcggtacacttgaaaaccgcggcagactcgtcagcgatgacgtgctgacgctcagtgccacgcagataaacaacagcggtaccctctccggggcaaaggaacttgtggcttctgcagacacactgaccaccacagaaaaatcggtcacaaacagtgacggtaacctcatgctggacagcgcgtcttccacactggcgggtgaaaccagtgcgggtggcacggtgtctgtaaaaggcaacagtctgaagaccacgaccactgcgcagacgcagggcaacagtgtcagcgtggatgtgcagaacgcacagcttgacggaacacaggctgccagagacatccttaccctgaacgccagtgaaaagctcacccacagcgggaaaagcagtgccccgtcgctcagcctcagtgcgccggaactgaccagcagcggcgtacttgttggttccgccctgaatacacagtcacagaccctgaccaacagcggtctgttgcaggggaaggcctcactcaccgttaacacacagaggcttgataatcagcagaacggcacgctgtacagtgctgcagacctgacgctggatataccggacatccgcaacagcgggcttatcaccggtgataatggtttaatgttaaatgctgtctccctcagcaatccgggaaaaatcatcgctgacacgctgagcgtcagggcgaccacgctggatggtgacggcctgttgcagggcgccggtgcactggcgcttgcaggtgacacactctcactgggtagtaacggacgctggctgacggcgggcgacctctccctccggggtaaaacactgcataccgcagggaccacgcagggacagaatctcaccgtgcaggcggacagatgggcgaacagtggttccgtgcaggcaaccggtaaccttactgcttcggcaaccggtcagttgaccagtaccggcgatatcatgagccagggtgacaccacgctgaacgcagccaccacggacaaccggggcagtctgctttcggccggcacgctctcccttgatggaaactcactggataacagcggcactgtccagggtaaccatgtcacgattcgccagaacggtgtcaccaacagtggcacgctcaccgggatcgccgcgctgacgcttgccgcccgtatggatatggcatcccctcaacctgcgctgatgaataacggaggttcattgctgaccagcggcgatctgacaatcaccgcaggcagtctggcaaacagcggggcgatccaggcggctgacagcctgactgcacgtctgacgggtgagctcgtcagcacagcaggcagcaaagtcacctcgaacggtgaaatggcgctcagtgcactgaatttaagcaacagcggacaatggattgcaaaaaatctgactctgaaggcgaactcactgaccagtgcgggtgacatcaccggtgtggatgctctcacgctcacggtgaatcagacgctgaacaatcacgcgagcggaaaactgctcagtgcaggtgtgctgacgctgaaggcagacagtgtcaaaaacgacgggcaattacagggaaatgccaccaccatcacggcaggacaactcacaaacggcgggcatctgcagggcgaaacgctgacgctggccgcctccggtggcgtgaacaaccgttccggtggtgttctgatgagccggaatgcactgaatgtcagtactgcgaccctgagtaaccagggcacgatacagggtggtggcggggtttccctgaacgccactgaccgtctgcagaacgacggcaaaatcctctccggcagtaacctgacgctgacggcgcaggtgctggcgaacaccggcagcggactggtacaggctgccaccctgctgctggatgtggtgaatactgtcaacggcggacgcgtacttgccaccggcagtgccgacgttaaaggaaccacgctgaataataccggtacgtttcagggggcggacctgctggtgaattaccacacattcagcaacagcggtaccctgctgggaacctccgggcttggcgtcaagggcagttcactgctgcaaaatggtacagggcggctgtacagtgcaggcaacctgctgcttgacgctcaggacttcagtggtcagggccaggtggtggccaccggtgatgtcacactgaaactgattgctgccctcacgaatcatggtaccctggccgcagggaaaaccctttccgtcacgtcgcagaacgccgtcaccaacggcggtgtgatgcagggtgatgccatggtgctcggtgccggagaggcattcaccaacaatggaacgctgactgccggtaaaggcaacagtgttttcagcgcacagcgtcttttccttaacgcaccgggttcacttcaggccggtggcgatgtgagtctgaacagccggagtgatatcaccatcagtggttttaccggcacggcaggcagtctgacaatgaatgtggccggtaccctgctgaacagtgcgctgatttatgcggggaataacctgaagctgtttacagaccgtctgcataaccagcatggtgatatcctggccggcaacagtctgtgggtacagaaggacagcagcggtacggcaaacagcgaaatcattaaccgttccggtaatattgaaaccacacgtggcgatatcaccatgaatacggcgcatctgctgaattcatgggatgccatttcggccagccatgaggtcattcctggaagcagtcacggggtgatttcaccggttccggaaaataaccggtggtggggtgttgtccggcatgatggtgtggaatatctggccgtgtactggggtaagggtgccacggtacctgatgaataccgtatccgtacgggagatacggaaacggtgacagtcagtgcctccggccatgctgccaggatatccggcggcgctgatatgcatatccgtgcaggacgactggataatgaggcatcgtttattctcgccggtggcggtatgaccctgagtggcgacacactgaacaatcagggctggcaggaggggaccaccggaaaggaaacggtctggcggcttgcctcaggctcactccccaaagcatggtttactgagccatggtataaggtctatcgccaggtctcaccggatgcgacagaagccagtggcacctctccggcaggtcagtaccgtgcggttatcagcgctgccggtgatgtatctgccagctttgccactgacaccggtaataccactgtcatgccccgggcaggtggtgcaggtaatacgataacggtgccttcgctgaactccctgacaccaccgacggtcagtcagggggtgagcggtgaagccctgctgaatgaaagcggtaccgggataaccggccctgtatggaatgacgcgctgccggatacgctgaaggatatcccgggtgcgctgtcgctgtccggggcaagtgtaagcagctatcccctgccatccggtaacaatggttattttgtcccgtccacggacccggacagtccgtatctgattacggtgaacccgaaactggatggtctcggaaaggtggacagcagtctgtttgccggactgtatgacctgctcaggatgcaacccggagaggcaccacgtgaaacagacccggcatataccgatgaaaaacagtttctgggctcgtcgtacattcttgaccgtcttggcctgaaaccggaaaaagattaccgctttctgggggatgcggcttttgatacccggtatgtcagtaacgtcatactgaaccagacgggctcacgttatatcaacggcacaggttctgacctggcgcagatgaaatacctgatggacagcgcggcagcacaacagaaagcgctcggactgacctttggtgtgtcgctgaccgccgggcaggttgcacaactgaccagaagcctgctgtggtgggagtccgtcaccatcaacggacagacagtcatggttccgaaactgtatctgtctccggaagatatcacccttcataatggcagcgttatcagcgggaacaacgtgcagcttgcgggtggcaatatcaccaacagcggcagcagcatcaacgcacagaatgacctcctgctcgacagaacaggtagtatcgacaacctgaatgccggactgataaacgccggtggtgcactgaacctgaaggccatcggggatatcggcaatatcagctcagtcatcagcggtaaaaccgtatccctggaaagcgccaccggaaacatcagtaatctcacccggacagaacagtgggcgatgaacaacggatataaccatttcagcggaacggataccggtccgctggctgctgtcagggcaacggactcactgtttatgggcgcagccggagatatcagtatcaccggtgccgccgtttctgccggtgacagcgtgttactggccgccggtaatgacctgaacatgaacgcgatacaggccggagaacgtcgtcgctatggcgggtccgggtggtatgaaactcatgccgtggcccccacggtcactgccgggaacagtctgatgctttccgccggtcgtgatgtgaacagccaggctgccggtattacagctgaaaacagtatggatatccgcgccggacgggatgtgaacatggctgcggagtccacaggcgcaggtgaccacgacagcactttcagcatgaaaacggttcatgattcggtccggcagcagggaacagacatgaccagcggcggtgacatcacggttactgccggacgggatatcacctcagttgcgactgctgtaacggcaaagggcgatatccgtgtgaatgccggtcatgacatcgtactgggtactgccacggaaagtgattatcactacagtgaatcaggggaaacccgtaaccgtctcctcagccatcagaccacccggaccatcacggaagacagcgtcacccgtgagaagggctccctgctgtcgggaaaccgcgtgaccgttaacgccggtaataacctgacggtacagggttcggatgtggtggctgaccgggatgtgtcactggcggcggataaccatgttgatgttcttgctgccaccagtacagatacgtcctggcgctttaaggaaacgaagacatccggtctgacgggtaccggcggtattggtttcaccactggcagcagtaagacaacgcacgaccgccgcgaggccgggacaacgcagagtcagagtgccagtaccatcggctccactgccggtaatgtcagtattaccgcgggcaaacaggctcatatcagcggttcggatgtgattgcgaaccgggatatcagcattaccggtgacagtgtggtggttgacccggggcatgaccgtcgtactgtggacgaaaaatttgagcagaagaaaagcgggctgacggttgccctgtccggtgcggtgggtagtgccatcaataatgcggtcaccatggcccgggaggcgaaagaaaccagcgacagccgtctggcggcactgaaaggaacacaggcggttctgtctggtgtacaggccggtgttaaccacggactgcagcaacagagtgctgacccgaataatggtatcggcgtgagcatttcgctcaaccatcagcagtcgaagtctgagacgaaatatcagcacgatatcgtctccggctcaaccctcagcgccggtaacaatgtctctgtcactgccacggggaaaaataaggaccataacaacagcggcgatatgctcattaccgggagccagattaagtccggcaatgacaccagcctgaatgcacagaacgatattctgctggctgccgccgctgacacccgacagacaaccgggaagaacagcagcaaaggtggcggcgtgggcgtcagctttggtggtggaaccaatggcggaggcctcagtatcttcgccggtatcaatggttcagagggcagggagaaaggcaacggcacgacctggaccgaaaccacgctggatgcgggtaaaaatgtctccctcacaagcggacgtgataccaccctgtcgggggctcaggtcagtggcgagaaagtcacagcagatgtcggcaacaacctgaccatttcgagccttcaggacagtgaccgttacgacagcagacagaaccgcgtggccgcaggtggcagcttcacctttggctcgatgtccggcagcggctatgccagcatcagccaggacaaaattaaaagcaattacgattctgtccgggaacagagtggtatctacgccggaaaagacgggtttgatgttactgtggggaatcacactcagctcaatggtgcggtaattgcgtccacggcaacggatgacaaaaacagcctcaataccaacacgctgggctggtcggatatccataaccaggcagactataaagccagccataccggaatcagtttatcgggtggctcagggatgtcagccagccagatggtggcctcaaatgcaatagccggtgcggcgaacgcacttaccggtatgtccggcagcagtggacatgctgaaggtaccacgtcatctgccatcagcggcggaaatctcatcatccgtaataaggagagtcagaagcaggatattgccgggctcagccgggacccggaaaacgccaacggcagcattgcaccgatatttgacagggagaaagagcagaagcgtcttcaggaagcgcaggttatcagccagattagtggtcagatgagtaacatcgtcatgacgtatggcgaaactgaagcgatgaaagcagccaggaaagaacatcctggcatgagcgatgcacagttgcgggagaccccggaatacagagaggtcatgaaaggctacggtacgggcagtacgccgcagatggtggtacaggccatcacgggcgtgctgggcggactgaatgcagggaatccggggcaggtactggctggcggtctgaatccggcagtggcacagcttataaaacaggctacaggcgataaccgggaagcaaacctgatggctcatgcagtctggggagcacttgccgcacaactgggcggaaacaatgcggcttccggtgctgccggggcattcagtggtgaacttgcagcccggtatatcattgataactactacggtggtcgtaccgataacctgagtgaacaggaacgtcagcaaatcagcatgctggccaccattgcttcaggtattgcaggcgggcttgtcggcaacagtacaagtgctgccggtacgggggcacaggcaggtaggaactcggttgagaataatgcaatgtctggcctggaagggtttgggaccggcttccagagttatgttcaggcccaggaggcgctggtaaataataccaatcttactgataaaaatggcaaagtactgaatccggcaacaccagaggaaattaagtacgcctctgataagctggtaaccggttctatacctgaaggacaggaccctgccagaggtttactgatttcatggggagccggagcatctgtttttgggggagaacttatagctccagcagttggcacggttgctgttatcggaggtaccctcttaggtggtactacagatgctgttaagcagtttttgaccctgaaaccgggtgagcaatacagtactacagatactcttattgccgcaggtgaaggtgggcttactcaggggaaaggggtcatcttcagtacatttattaacacaatgggggcttacttaggaagtaaagctaaaggggaagaccctacagggccaatggtgggaaatgctataggtactgcactgggaaataaagccggagataaattcacaaaagaaatgctctcaagaggctttggctctgtcacatcagaggttacaggtacagttactggtagtgttattgggacagtgactgattatcagatcgaaaagctaggaaaaggtaataaggaaggggcaaaatga

SEQ ID NO: 3 - DNA, 240 bp, Escherichia coli
  misc_feature (1)..(240): Sufficient for immunity to contact-dependent inhibition
atgaagaagaaactatttgcccttttaaaatatattattttttttcctatgctatgtactgtacttggtcttttaggtatacctataggtttaattgtgaattttttgagaacgggctcttttgacttcaacttaaaagatgaaattgatgttgttttattcactttaaaaataggaatccccataggctttattcttgggctgggtttgtggggccttagtatcttagacagaaaatag

SEQ ID NO: 4 - protein, 588 aa, Escherichia coli
  PEPTIDE (1)..(588): Protein of cdiB
MRHRQDNLLANRTLLPGMASGQYVFRLCTFSPVVRYFSLLPCLCILSFSSPAAMLSPGDRSAIQQQQQQLLDENQRQRDALERSAPLTITPSPETSAGTEGPCFTVSSIVVSGATRLTSAETDRLVAPWVNQCLNITGLTAVTDAMTDSYIRRGYITSRAFLTEQDLSGGVLHITVMEGRLQQIRAEGADLPARTLKMVFPGMEGKVLNLRDIEQGMEQINRLRTEPVQIEISPGDREGWSVVTLTALPEWPVTGSVGIDNSGQKSTGTGQLNGVLSFNNPLGLADNWFVSGGRSSDFSVSHDARNFAAGVSLPYGYTLVDYTYSWSDYLSTIDNRGWRWRSTGDLQTHRLGLSHVLFRNGDMKTALTGGLQHRIIHNYLDDVLLQGSSRKLTSFSVGLNHTHKFLGGVGTLNPVFTRGMPWFGAESDHGKRGDLPVNQFRKWSVSASFQRPVTDRVWWLTSAYAQWSPDRLHGVEQLSLGGESSVRGFKDQYISGNNGGYLRNELSWSLFSLPYVGTVRAVAALDGGWLHSDSDDPYSSGTLWGAAAGLSTTSGHVSGSFTAGLPLVYPDWLAPDHLTVYWRVAVAF

SEQ ID NO: 5 - protein, 3132 aa, Escherichia coli
  PEPTIDE (1)..(3132): Protein of cdiA
MHQPPVRFTYRLLSYLVSAIIAGQPLLPAVGAVITPQNGAGMDKAANGVPVVNIATPNGAGISHNRFTDYNVGKEGLILNNATGKLNPTQLGGLIQNNPNLKAGGEAKGIINEVTGGKRSLLQGYTEVAGKAANVMVANPYGITCDGCGFINTPHATLTTGKPVMNADGSLQALEVTEGSITINGAGLDGTRSDAVSIIARATEVNAALHAKDLTVTAGANRITADGRVSALKGEGNVPKVAVDTGALGGMYARRIHLTSTESGVGVNLGNLYAREGDIILSSSGKLVLKNSLAGGNTTVTGTDVSLSGDNKAGGNLSVTGTTGLTLNQSRLVTDKNLVLSSSGQIVQNGGELTAGQNAMLSAQHLNQTSGTVNAAENVTLTTTDDTTLKGRSVAGKTLTVSSGSLNNGGTLVAGRDATVKTGTFSNTGTVQGNGLKVTATDLTSTGSIKSGSTLDISARNATLSGDAGAKDRALVTVSGTLENRGRLVSDDVLTLSATQINNSGTLSGAKELVASADTLTTTEKSVTNSDGNLMLDSASSTLAGETSAGGTVSVKGNSLKTTTTAQTQGNSVSVDVQNAQLDGTQAARDILTLNASEKLTHSGKSSAPSLSLSAPELTSSGVLVGSALNTQSQTLTNSGLLQGKASLTVNTQRLDNQQNGTLYSAADLTLDIPDIRNSGLITGDNGLMLNAVSLSNPGKIIADTLSVRATTLDGDGLLQGAGALALAGDTLSLGSNGRWLTAGDLSLRGKTLHTAGTTQGQNLTVQADRWANSGSVQATGNLTASATGQLTSTGDIMSQGDTTLNAATTDNRGSLLSAGTLSLDGNSLDNSGTVQGNHVTIRQNGVTNSGTLTGIAALTLAARMDMASPQPALMNNGGSLLTSGDLTITAGSLANSGAIQAADSLTARLTGELVSTAGSKVTSNGEMALSALNLSNSGQWIAKNLTLKANSLTSAGDITGVDALTLTVNQTLNNHASGKLLSAGVLTLKADSVKNDGQLQGNATTITAGQLTNGGHLQGETLTLAASGGVNNRSGGVLMSRNALNVSTATLSNQGTIQGGGGVSLNATDRLQNDGKILSGSNLTLTAQVLANTGSGLVQAATLLLDVVNTVNGGRVLATGSADVKGTTLNNTGTFQGADLLVNYHTFSNSGTLLGTSGLGVKGSSLLQNGTGRLYSAGNLLLDAQDFSGQGQVVATGDVTLKLIAALTNHGTLAAGKTLSVTSQNAVTNGGVMQGDAMVLGAGEAFTNNGTLTAGKGNSVFSAQRLFLNAPGSLQAGGDVSLNSRSDITISGFTGTAGSLTMNVAGTLLNSALIYAGNNLKLFTDRLHNQHGDILAGNSLWVQKDSSGTANSEIINRSGNIETTRGDITMNTAHLLNSWDAISASHEVIPGSSHGVISPVPENNRWWGVVRHDGVEYLAVYWGKGATVPDEYRIRTGDTETVTVSASGHAARISGGADMHIRAGRLDNEASFILAGGGMTLSGDTLNNQGWQEGTTGKETVWRLASGSLPKAWFTEPWYKVYRQVSPDATEASGTSPAGQYRAVISAAGDVSASFATDTGNTTVMPRAGGAGNTITVPSLNSLTPPTVSQGVSGEALLNESGTGITGPVWNDALPDTLKDIPGALSLSGASVSSYPLPSGNNGYFVPSTDPDSPYLITVNPKLDGLGKVDSSLFAGLYDLLRMQPGEAPRETDPAYTDEKQFLGSSYILDRLGLKPEKDYRFLGDAAFDTRYVSNVILNQTGSRYINGTGSDLAQMKYLMDSAAAQQKALGLTFGVSLTAGQVAQLTRSLLWWESVTINGQTVMVPKLYLSPEDITLHNGSVISGNNVQLAGGNITNSGSSINAQNDLLLDRTGSIDNLNAGLINAGGALNLKAIGDIGNISSVISGKTVSLESATGNISNLTRTEQWAMNNGYNHFSGTDTGPLAAVRATDSLFMGAAGDISITGAAVSAGDSVLLAAGNDLNMNAIQAGERRRYGGSGWYETHAVAPTVTAGNSLMLSAGRDVNSQAAGITAENSMDIRAGRDVNMAAESTGAGDHDSTFSMKTVHDSVRQQGTDMTSGGDITVTAGRDITSVATAVTAKGDIRVNAGHDIVLGTATESDYHYSESGETRNRLLSHQTTRTITEDSVTREKGSLLSGNRVTVNAGNNLTVQGSDVVADRDVSLAADNHVDVLAATSTDTSWRFKETKTSGLTGTGGIGFTTGSSKTTHDRREAGTTQSQSASTIGSTAGNVSITAGKQAHISGSDVIANRDISITGDSVVVDPGHDRRTVDEKFEQKKSGLTVALSGAVGSAINNAVTMAREAKETSDSRLAALKGTQAVLSGVQAGVNHGLQQQSADPNNGIGVSISLNHQQSKSETKYQHDIVSGSTLSAGNNVSVTATGKNKDHNNSGDMLITGSQIKSGNDTSLNAQNDILLAAAADTRQTTGKNSSKGGGVGVSFGGGTNGGGLSIFAGINGSEGREKGNGTTWTETTLDAGKNVSLTSGRDTTLSGAQVSGEKVTADVGNNLTISSLQDSDRYDSRQNRVAAGGSFTFGSMSGSGYASISQDKIKSNYDSVREQSGIYAGKDGFDVTVGNHTQLNGAVIASTATDDKNSLNTNTLGWSDIHNQADYKASHTGISLSGGSGMSASQMVASNAIAGAANALTGMSGSSGHAEGTTSSAISGGNLIIRNKESQKQDIAGLSRDPENANGSIAPIFDREKEQKRLQEAQVISQISGQMSNIVMTYGETEAMKAARKEHPGMSDAQLRETPEYREVMKGYGTGSTPQMVVQAITGVLGGLNAGNPGQVLAGGLNPAVAQLIKQATGDNREANLMAHAVWGALAAQLGGNNAASGAAGAFSGELAARYIIDNYYGGRTDNLSEQERQQISMLATIASGIAGGLVGNSTSAAGTGAQAGRNSVENNAMSGLEGFGTGFQSYVQAQEALVNNTNLTDKNGKVLNPATPEEIKYASDKLVTGSIPEGQDPARGLLISWGAGASVFGGELIAPAVGTVAVIGGTLLGGTTDAVKQFLTLKPGEQYSTTDTLIAAGEGGLTQGKGVIFSTFINTMGAYLGSKAKGEDPTGPMVGNAIGTALGNKAGDKFTKEMLSRGFGSVTSEVTGTVTGSVIGTVTDYQIEKLGKGNKEGAK

SEQ ID NO: 6 - protein, 79 aa, Escherichia coli
  PEPTIDE (1)..(79): Protein for cdiI
MKKKLFALLKYIIFFPMLCTVLGLLGIPIGLIVNFLRTGSFDFNLKDEIDVVLFTLKIGIPIGFILGLGLWGLSILDRK

(19) United States
(12) Patent Application Publication
(10) Pub. No.: US 2008/0317762 A1
(43) Pub. Date: Dec. 25, 2008

(54) Bacterial Contact Inhibition and Methods Therefor

(76) Inventors: Stephanie Aoki, Goleta, CA (US); Rupinderjit Pamma, Sacramento, CA (US); Bruce Braaten, Goleta, CA (US); Aaron Hernday, Goleta, CA (US); Jessica Erin Bickham, Santa Barbara, CA (US); David A. Low, Goleta, CA (US)

Correspondence Address: FISH & ASSOCIATES, PC;ROBERT D. FISH, 2603 Main Street, Suite 1050, Irvine, CA, 92614-6232, US

(21) Appl. No.: 12/063,902

(22) PCT Filed: Aug. 16, 2006
(86) PCT No.: PCT/US2006/032064

§ 371 Date: Jul. 25, 2008

Related U.S. Application Data
(60) Provisional application No. 60/709,407, filed on Aug. 17, 2005.

(51) Int. Cl. A61K 39/395; C07H 21/00; C12N 15/63; C12N 1/21; A61P 31/04; C07K 14/00; C12N 1/20

(52) U.S. Cl. 424/164.1; 536/23.7; 435/320.1; 435/252.3; 530/350; 530/324; 435/252.1

(57) ABSTRACT

Contemplated compositions and methods relate to bacterial contact inhibition that is conferred by two gene products, CdiA and CdiB, which are encoded by the genes cdiA and cdiB, respectively. A further gene product CdiI, encoded by the gene cdiI confers immunity to contact inhibition caused by CdiAB.

This application claims priority to our copending U.S. provisional patent application with the Ser. No. 60/709407, which was filed Aug. 17, 2005.

This invention was made with government support under grant NIH AI23348 from the National Institutes of Health. The government may have certain rights in the invention.

### FIELD OF THE INVENTION

The field of the invention is antimicrobial agents, especially as they relate to bacterial contact inhibition.

### BACKGROUND OF THE INVENTION

Contact inhibition is a common regulatory phenomenon observed in many eukaryotic cells and most typically involves direct cell-to-cell contacts. In many cases, contact inhibition is a critical determinant for growth control and/or density regulation (e.g., J. Membr. Biol. 1981;63 (1-2):1-11). Thus, and not surprisingly, defects in contact inhibition are frequently observed in various diseases or dysfunctional states (see e.g., Bioessays 1993 Dec;15(12): 807-13).

Remarkably, while contact inhibition can be observed throughout many species in higher organisms, contact inhibition among bacteria has to the best of the inventors' knowledge not been reported. Only in rare cases, selected bacteria were shown to interfere with contact inhibition of non-bacterial cells. For example, Hep2 cells tend to lose contact inhibition when cultured with Helicobacter spec under certain conditions (see Mikrobiol. Z. 2003 Jul-Aug;65(4):11-6), and kidney cells loose contact inhibition under some conditions when cultured with Listeria (see e.g., Biol Cell. 1995;85(1):55-66). Bacterial growth and/or division is typically inhibited using one or more antibiotic drugs that either block metabolism and/or interfere with cell wall growth or integrity. However, increasing use of antibiotics prompted emergence of resistant forms at a relatively fast pace.

Thus, it would be desirable to have a new strategy to interfere with bacterial growth and growth arrest in a manner that is independent of the mechanisms of action of currently known antibiotics. Consequently, there is still a need to provide new compositions and methods to target bacterial growth or growth arrest.

### SUMMARY OF THE INVENTION

The present invention is directed to compositions and methods related to bacterial contact inhibition. Most preferably, such contact inhibition is conferred by expression of two genes, cdiA and cdiB, and immunity to contact inhibition is conferred by expression of one gene, cdiI.

In one aspect of the inventive subject matter, an isolated nucleic acid sequence that encodes a peptide of a bacterial contact inhibition system has a sequence selected from the group consisting of SEQ. ID. No. 1, SEQ. ID. No. 2, and SEQ. ID. No. 3, or has a sequence that has at least 70% identity with SEQ. ID. No. 1, SEQ. ID. No. 2, and SEQ. ID. No. 3, respectively. Such sequences may also be truncated, comprise insertions, 5′-additions, and/or 3′-additions, and are preferably cloned in a vector to trigger expression of the recombinant sequence in vitro and/or in vivo. Consequently, bacterial cells comprising such sequences and vectors that include such sequences are also contemplated.

In another aspect of the inventive subject matter, an isolated peptide of a bacterial contact inhibition system has a sequence selected from the group consisting of SEQ. ID. No. 4, SEQ. ID. No. 5, and SEQ. ID. No. 6, or a homolog thereof having at least 80% identity with SEQ. ID. No. 4, SEQ. ID. No. 5, and SEQ. ID. No. 6, respectively. Such polypeptides are preferably recombinant and encoded by the above DNA sequences, and may further include affinity—or other tags.

In still another aspect of the inventive subject matter, a method of reducing bacterial contact inhibition comprises a step of contacting the bacterium with a compound that binds to at least one component of the bacterial contact inhibition system at a concentration effective to reduce bacterial contact inhibition. Preferably, the compound is an antibody or fragment thereof (e.g., isolated monoclonal antibody) that specifically binds to protein having a sequence of SEQ. ID. No. 4 or SEQ. ID. No. 5 (or homologous sequence).

Consequently, a method of treating a chronic bacterial infection will therefore include a step of ascertaining that a bacterium (e.g., Escherichia coli, Yersinia pestis, Burkholderia pseudomallei, etc.) expresses a nucleic acid that encodes for a component of a bacterial contact inhibition system. In anther step, instructions are provided to contact the bacterium with a compound (e.g., antibody or a fragment thereof) that binds to at least one component of the bacterial contact inhibition system at a concentration effective to reduce bacterial contact inhibition. Then, where desired, instructions are provided to contact the bacterium with an antibiotic drug.

Various objects, features, aspects and advantages of the present invention will become more apparent from the following detailed description of preferred embodiments of the invention, along with the accompanying drawing.

### BRIEF DESCRIPTION OF THE DRAWING

FIGS. 1A and 1B are graphs depicting growth inhibition of E. coli K12 strains by wild-type Escherichia coli isolate EC93.

FIGS. 2A-2E are graphs depicting various results from a FACS analysis using differently labeled cells.

FIGS. 3A-3C illustrate results from FACS analysis using a cdiA FLAG-mutants (A and B) and results from complementation analysis with a functional homolog of cdiB (C).

FIG. 4A-4D are graphs depicting dependence of contact dependent inhibition on cellular growth phase.

FIG. 5 is a graph depicting results from a chloramphenicol pretreatment/inhibition recovery assay.

FIG. 6 is a graph depicting results of contact dependent inhibition under different inhibitor to target cell ratios.

FIG. 7A and 7B are graphs depicting results indicating lack of release of growth inhibitory activity into the growth medium.

FIG. 8 is a graph depicting results of PET membrane control experiments

FIG. 9A and 9B are graphs depicting results of an analysis of immunity regions linked to cdiAB.

FIG. 10 is a graph depicting results of a complementation assay using the cdiA homologue from UPEC strain 536.

### DETAILED DESCRIPTION

The inventors have surprisingly discovered that various bacteria exhibit pronounced contact inhibition on other bacteria when allowed to contact the other bacteria. More specifically, the inventors discovered that bacteria capable of inhibiting growth of other bacteria have a nucleic acid encoding for at least two, and in some cases three peptides, wherein at least a portion of two of the three peptides are located on the exterior membrane of the bacterium, and wherein the third of the peptides confers immunity to that bacterium against contact inhibition where the other two peptides are expressed. Remarkably, while certain bacteria secrete small peptide bacteriocins (e.g., colicins and microcins), which kill target bacteria, for example by forming membrane pores, the peptides according to the present inventive subject matter appear to act as true inhibitors of cell growth and/or metabolism and require contact with a target cell to inhibit the cell.

In one particularly contemplated example, the inventors identified a system by which one bacterium (here: Escherichia coli K-12) can inhibit another bacterium (here: Escherichia coli K12) through direct cell-to-cell contact, wherein the inhibiting bacterium has cdiAB genes derived from wild-type Escherichia coli strains including uropathogenic Escherichia coli (CDI: contact-dependent inhibition). The inventors further discovered that the cdiAB genes have homologues in other pathogens, including Yersinia pestis (etiologic agent for plague) and Burkholderia pseudomallei (etiologic agent for melioidosis). Remarkably, some E. coli also contain an immunity protein, which the inventors designated CdiI, that confers full protection against growth inhibition to cells making the CdiA/B gene products. Without this immunity function, the expression of CdiAB would inhibit cell growth.

It should be particularly noted that certain urinary tract pathogens do not appear to have any immunity function but do have the cdiA/B genes. This suggests, among other things, that the cdiA/B system will place the bacteria into a quiescent or low metabolic activity state under certain growth conditions, and thus may contribute to chronic disease. Viewed from another perspective, the cdiA/B system may present a switch, that under particular growth conditions, will put at least a fraction of a bacterial population into a quiescent growth state.

Interestingly, a quiescent form of uropathogenic Escherichia coli (UPEC) was previously reported within a host cell (bacteria were present, apparently intact, but could not be cultured), and it was hypothesized that these forms would provide a reservoir of cells that are not susceptible to classic antibiotic treatment, and that are therefore a source of recurrence once the antibiotic treatment ended. Among others, Scott Hultgren at Wash U St Louis has shown that UPEC can form “pods” inside bladder cells in which the bacteria form a colony mass similar to a biofilm inside the cell, and that some of these cells become non-culturable (i.e., will not grow on laboratory media) but may still be alive. In such a state the bacteria may be able to hide from the immune system and avoid killing by antibiotics, which relates perhaps to the observation that many patients with chronic urinary tract infections have repeated outbreaks of the same bacteria even after multiple antibiotic treatments.

Based on this and other (see below) observations, it should then be recognized that the cdiAB system may provide a novel target for antimicrobial treatment. Among other things, it is contemplated that if the CDI system is blocked, bacteria should be prevented from entering the quiescent state and thus become/remain susceptible to antibiotic treatment. Consequently, under such a scenario, a patient may receive an antibiotic plus a drug that blocks the CDI system, which should prevent growth of all bacteria present in the patient.

In one aspect of the inventive subject matter, the inventors contemplate all nucleic acids that encode cdiA, cdiB, cdiI, and all functional homologs and analogs thereof. Specific examples for cdiA, cdiB, cdiI sequences are given in the attached pages. Of course, it should be recognized that suitable sequences can be modified in a random or specific manner to achieve a particular purpose (e.g., adjust codon usage, generate specific mutants, add restriction site, etc.), or that insertion, deletion, or other mutations can be performed for a desired outcome (e.g., adding His-tags, making dimers, etc.). Suitable nucleic acid sequences may be present as DNA, RNA, PNA, or otherwise chemically modified nucleic acid as appropriate. Therefore, homologs of these sequence having at least 70%, more preferably 80%, even more preferably 90%, and most preferably 95% identity are contemplated.

Furthermore, it should be recognized that the nucleic acid may be cloned into a vector to manipulate, or amplify a particular sequence, or to achieve a functional purpose (e.g., recombinant expression of gene product or anti-sense RNA) in a prokaryotic or eukaryotic cell. There are numerous vectors known in the art, and all of them are considered suitable for use herein. Consequently, it should be recognized that pro- and/or eukaryotic cells may be transfected with various constructs carrying one or more components of the CDI system. Such cells may particularly serve in the identification of the molecular mechanism of the CDI system, to identify the target of the CdiA/B component, and/or as therapeutic modality (see below).

In further aspects of the inventive subject matter, all sequences (nucleic acid and/or peptide) are contemplated that confer ability to provide contact inhibition and/or immunity thereto, and especially suitable sequences will have at least 60% homology, and more typically at least 70%-80%, and even more typically at least 80%-95% homology to the sequences presented herein. However, it is not necessarily required that the analog or homolog sequences are identical or homolog throughout the entire length, but that only critical domains will have a significant degree (at least 70%) of homology and/or identity. For example, Y. pestis and B. pseudomallei homologues of CdiA are only 24-30% identical to the E. coli protein, yet share blocks of conserved amino acids that are thought to act as functional homologues.

In particularly contemplated aspects, it should be appreciated that the CDI system can advantageously be used to treat bacterial infections by either providing immunity, blocking immunity, providing contact inhibition, blocking contact inhibition, and/or by interference with the receptor of the cdiA/B system in a target cell to either render the target cell immune to the CDI system, or to trigger the quiescent state in the target cell.

For example, it is contemplated that entry into a quiescent state can be prevented by providing cdiI to the target cells, and/or by blocking or reduction of expression of the cdiA/B genes in the donor cell. Alternatively, or additionally, compositions may be provided to the donor cell that render the CdiA/B component(s) inoperable (e.g., small fragments of the CdiA/B ligand, small-molecule drugs binding to the CdiA/B component(s), or antibodies against the Cdi A/B component(s). In further contemplated aspects, it should also be recognized that the natural ligand of the CdiA/B may be targeted, using similar types of compounds as outlined above. Additionally contemplated therapeutic approaches include those in which a non-pathogenic bacterium is transfected with a vector that expresses CdiI to thereby provide in situ immunity to a cell that would otherwise enter quiescence.

On the other hand, where it is desired that a (pathogenic) cell should enter a quiescent state, it is generally contemplated that such entry can be promoted with transfected non-pathogenic bacteria expressing the CdiA/B system, or with formulations comprising the CdiA/B components. Among other formulations, it is generally preferred (but not necessarily so) that the CdiA/B containing formulation comprises a lipid component. For example, the CdiA/B components may be present in form of a liposome, a partially purified membrane fraction, an emulsion, etc.

Thus, and among other useful therapeutic approaches, it is contemplated that such treatments may be suitable to prevent chronic urinary tract infections and also other chronic diseases (e.g., melioidosis) that are potentially associated with a quiescent state of a pathogen. With respect to treatment of many other bacterial diseases, it is contemplated that all diseases are suitable so long as the bacteria causing such diseases are inhibited by the CDI system. Such inhibition may be within a species, or may cross species barriers depending on the particular donor/recipient combination. However, where incompatibility is observed, genetic manipulation of the CdiA/B components may be helpful to restore compatibility.

Quantitative and qualitative analysis of the expression level of the components of the CDI system may be employed in an analytic and/or diagnostic manner in which the component(s) is/are detected in vivo or in vitro using methods well known in the art. For example, peptide expression may be monitored using ELISA, Western blot, or immunoscintigraphy. In another example, peptide expression may be quantified ex vivo from biopsy specimen using FISH or other immunographic methods. Where nucleic acid detection is desired, all known hybridization and/or amplification methods are deemed appropriate. Contemplated diagnostic applications also include nucleic acid and/or peptide sequence determination to identify and/or characterize mutations that may be present. The outcome of such analysis may then determine the proper course of therapy.

Based on the inventors' discovery of the implication of the cdiA/B and cdiI genes and gene products in bacterial contact inhibition, it is generally contemplated that all compounds that directly or indirectly interfere with the cdiA/B and cdiI genes and/or gene products are deemed suitable herein. The inventors therefore contemplate a method of reducing bacterial contact inhibition comprising a step of contacting the bacterium with a compound that binds to at least one component (CdiA and/or CdiB) of the bacterial contact inhibition system at a concentration effective to reduce bacterial contact inhibition. For example, where the nucleic acids are targeted, it is contemplated that suitable compounds include antisense and siRNA to reduce or even eliminate expression of one or more components of the CDI system. Thus, depending on the desired outcome, immunity or the capability to inhibit other cells may be destroyed. In another example, where the gene products are targeted, it should be recognized that all agents that bind to one or more components of the CDI system are contemplated. For example, antibodies, fragments of the CDI system, or small-molecule ligands may be obtained the will bind in a selective manner to the CDI system component(s). Consequently, antibodies against (or other molecules targeting) the CdiA/B protein may abolish capability of contact inhibition.

Similarly, it should be recognized that the physiological target of the CDI system in other cells may also be targeted. Most preferably, such approaches will make use of the CdiA/B system ‘receptor’ in a manner such that the receptor is blocked without triggering entry of the bacterium into the quiescent state. On the other hand, where desired, molecules may also be designed that bind to the receptor and promote entry into the quiescent state. In still further contemplated aspects, such molecules may be provided to the cell, host cell, or patient in isolated form, or associated with a membrane-like carrier (e.g., on or in a liposome) to facilitate contact with the bacteria. Finally, it should further be appreciated that components of the pathway or pathways that are functionally associated with the target of the CDI system may be identified, which will thus open additional therapeutic approaches for treatment of bacterial infections.

Therefore, the inventors also contemplate a method of treating a chronic or recurring bacterial infection in which it is first ascertained (e.g., via PCR, cell culture, etc.) that a bacterium (e.g., Escherichia coli, Yersinia pestis, and Burkholderia pseudomallei) expresses a nucleic acid that encodes for a component of a bacterial contact inhibition system. If such expression is confirmed, an instruction (e.g., printed, verbal, displayed, or otherwise) may then be given to contact the bacterium with a compound that binds to at least one component of the bacterial contact inhibition system at a concentration effective to reduce bacterial contact inhibition. Where appropriate, instructions may then be provided to contact the bacterium with an antibiotic drug (e.g., beta-lactam antibiotic, cephalosporin antibiotic, tetracycline antibiotic, macrolide antibiotic, and/or fluoroquinoline antibiotic).

It should be recognized that suitable compounds for targeting the CDI system and associated pathways can be obtained using immunology, combinatorial chemistry, and recombinant DNA technology, all of which are well known in the art. For example, antibodies can be generated from recombinant CdiA/B, while CdiA/B ligands may be obtained via affinity chromatography of membrane fractions of target bacteria.

### Experiments

The following experiments support the inventors' finding that various bacteria, and especially various Escherichia coli strains, including uropathogenic strains, contain a bacterial growth-inhibition system that uses direct cell-to-cell contact. Inhibition is typically conditional, dependent upon the growth state of the inhibitory cell and the pili expression state of the target cell. Both a large cell-surface protein designated Contact-dependent inhibitor A (CdiA) and two-partner secretion family member CdiB were required for growth inhibition. The CdiAB system is therefore thought to function as growth regulator of specific cells within a differentiated bacterial population.

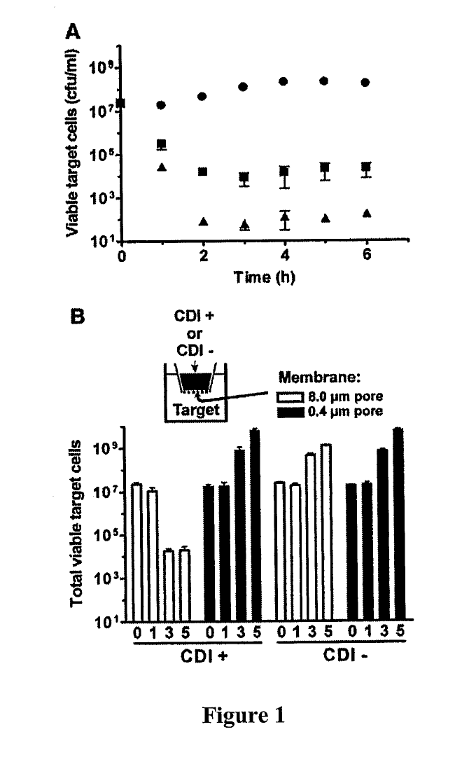

Wild-type Escherichia coli isolate EC93 inhibited the growth of laboratory E. coli K12 strains, such as MG1655, when the bacteria were mixed together in shaking liquid culture (FIG. 1A). Here, target E. coli MG1655 tet^{R} cells were mixed with the following test strains: E. coli EC93 inhibitory cells (squares); E. coli K-12 EPI100 containing pDAL660Δ1-39 (cdiA⁺B⁺, triangles); and control E. coli K-12 EPI100 str^{R} (circles). The inhibitor-to-target ratio for these experiments was 10:1. At the times indicated, viable target cell counts were obtained. In contrast, E. coli K-12 strains in general (e.g., EPI100) did not exhibit growth-inhibitory activity (FIG. 1A). It was further found that isolated genes from strain EC93, cdiA and cdiB, when expressed in E coli K-12 conferred a growth-inhibitory phenotype (FIG. 1A). Growth inhibition was dependent upon the growth state of the inhibitory cells, occurring in logarithmic but not stationary phase.

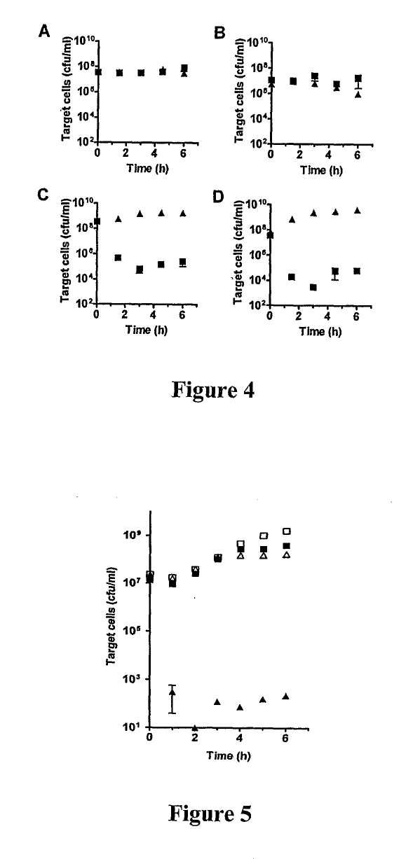

Target cells, however, were inhibited regardless of their growth phase (FIGS. 4A-4D). Here, E. coli CDI+EC93 inhibitor cells and E. Coli MC4100 (K-12) CDI-control cells were separately grown in LB medium to stationary phase (16 h) or logarithmic phase (OD₆₀₀ 0.35). EC93 and MC4100 cells were then mixed with E. coli MC4100 nal target cells and incubated with shaking (225 rpm) for various times. The viability of target E. coli grown in the presence of EC93 inhibitory cells (squares) or CDI-minus MC4100 control cells (triangles) is plotted on the y-axis. Panel A depicts stationary phase inhibitor or control cells incubated with stationary phase target cells (inhibitor or control to target cell ratio of 500/1). Panel B depicts stationary phase inhibitor or control cells incubated with logarithmic phase target cells (inhibitor or control to target cell ratio of 500/1). Panel C depicts logarithmic phase inhibitor or control cells incubated with stationary phase target cells (inhibitor or control to target cell ratio of 5/1), and panel D depicts logarithmic phase inhibitor or control cells incubated with logarithmic phase target cells (inhibitor or control to target cell ratio of 5/1).

Protein synthesis also appeared to be required for inhibition, because cdiA⁺B⁺ E. coli inhibitor cells pretreated for 2 hours with chloramphenicol did not have measurable inhibitory activity (FIG. 5). Here, E. coli EPI100 CDI+ (DL4577) and EPI100 CDI-minus control cells containing vector only (DL4527) cells were separately grown to logarithmic phase in LB-amp medium (OD₆₀₀ 0.35). Cultures were pretreated with chloramphenicol (34 μg/ml) for 2 h before adding E. coli DL4793 target cells (16 h) at a 10/1 inhibitor or control to target ratio. An untreated (no chloramphenicol) competition was included as a positive control for the inhibition of target cells. Competitions were incubated at 37° C. with shaking (225 rpm) and viable target cell counts were determined at the times indicated. Target cells incubated with CDI+ inhibitory cells are shown by triangles and target cells incubated with CDI-control cells are shown by squares. Cam pretreatment is represented by open symbols and the no treatment control is represented by filled symbols.

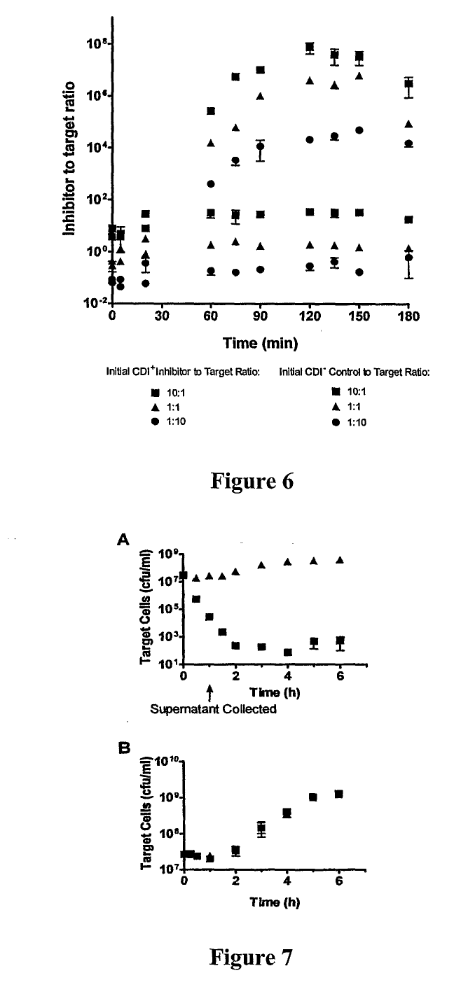

Experiments measuring the inhibitor-to-target ratio over time (FIG. 6) indicate that one cdiA⁺B⁺ E. coli cell inhibited the growth of multiple target cells. Starting at an initial ratio of 1 inhibitor to 10 target cells, after 1 hour this ratio increased more than a thousand-fold (FIG. 6). More specifically, E. coli CDI+ inhibitor (DL4577) cells were grown to logarithmic phase (OD₆₀₀=0.35). Inhibitor cells (15 mL) were added to flasks (125 ml) and inoculated with DL4793 target cells (16 h culture) at the inhibitor to target cell ratios indicated. Mixed cultures were incubated at 37° C. with shaking (150 rpm) and viable counts of inhibitory and target cells were obtained and plotted as a ratio at the times indicated. CDI-strain DL4527 was used in place of DL4577 as a negative control for inhibitory activity. In contrast, a control experiment with cdiAB-negative cells in place of cdiA⁺B⁺ inhibitory cells showed that the control-to target ratio was not significantly altered over the 3-hour time course (FIG. 6).

The growth-inhibitory activity did not appear to be a colicin, which is a secreted antimicrobial peptide, because supernatant solutions from logarithmic phase EC93 cultures lacked inhibitory activity even when a known inducer of colicin synthesis, mitomycin C, was present. To test the possibility that induction of inhibitory activity might occur only when target cells are present, the inventors prepared conditioned medium from a mixed culture of cdiA⁺B⁺ inhibitor cells and E. coli K-12 target cells (with a 10:1 inhibitor-to target ratio).

Target cell viability was reduced more than a thousand-fold after 1 hour of incubation (FIG. 7A), indicating substantial growth inhibitory activity. In this experiment, E. coli EPI100 CDI+ (DL4577) inhibitor and EPI100 CDC″ (DL4527) inhibitor control cells were separately grown to logarithmic phase (0D600—=, 0.35) in LB-amp medium. Target MG1655 tetR (DL4793) cells were added at a 10/1 inhibitor or control cell to target cell ratio. Viable DL4793 target cell counts obtained after addition of CDI+ inhibitor (squares) or CDI-inhibitor control (triangles) cells for the times indicated are shown. Conditioned medium from this 1-hour mixture, when added to fresh K-I12 target cells, did not affect cell growth compared to conditioned medium from control cdiA⁻B⁻ cell mixtures (FIG. 7B). To that effect, at the one hour time point in panel A above (indicated by arrow), a 15 ml sample from each culture was centrifuged at 6000×g for 1 min and supernatant solutions were filtered through a low protein binding 0.45 um HT Tuffryn membrane (Pall Life Sciences) to remove any remaining cells. A stationary phase culture (16 h) of target E. coli DL4793 was added 1/200 (v/v) into 12.5 ml of filtered cell culture supernatant, and viable target cell counts were determined at the times indicated. Results obtained after addition of filtered supernatants of CDI+ mixed cultures (squares) or CDI mixed cultures (triangles) are shown.

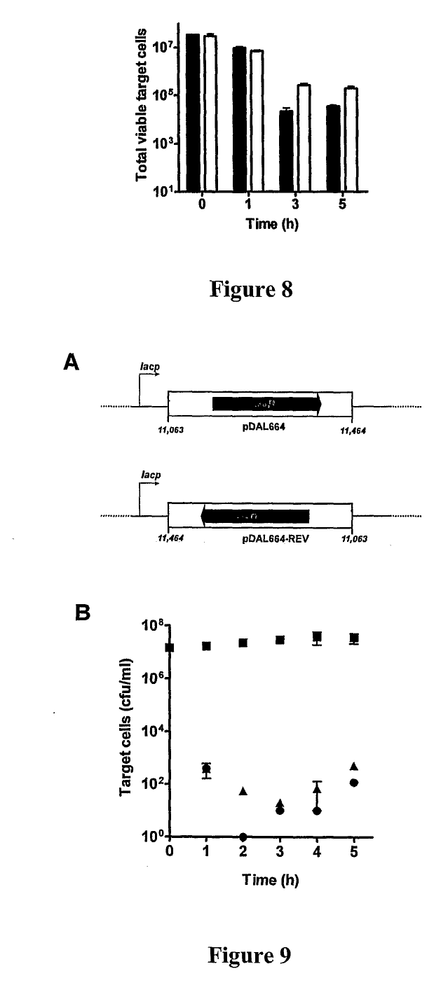

The inventors tested the possibility that growth inhibition might occur through cell-to-cell contact by separating cdiA⁺B⁺ inhibitory cells from target cells, using polyethylene terephthalate (PET) porous membranes in a six-well plate (FIG. 1B). Here, E. coli CDI⁺ K-12 (DL4577) or CDI⁻ E. coli K-12 (DL4527) were grown to logarithmic phase and added to the top chamber of a six-well plate containing either a 0.4-um (solid bars) or a 8 μm (open bars) PET membrane. Target E. coli MG1655 tet^{R} cells were added to the bottom well (in a 20:1 inhibitor-to-target ratio). Viable counts for the top and bottom chambers were measured after incubation (hours). Growth inhibition was not observed when contact between inhibitory cells and target cells was blocked by 0.4-μm pores; however, growth inhibition (˜1000-fold) was observed when 8-μm pores were used, allowing inhibitor and target cell mixing. Addition of cdiA−B− negative control E. coli did not affect growth of target cells, regardless of pore size (FIG. 1B). The inventors obtained similar results using EC93, from which the cdiAB genes were isolated for cloning into E. coli K-12. To test whether a secreted inhibitory molecule would bind to the PET membrane and be sequestered or inactivated, excess PET membranes were added to the cultures, which did not affect the ability of cdiA+B+ E. coli K-12 to inhibit cell growth (FIG. 8). Here, PET membranes were minced aseptically with dissecting scissors and added to top wells of a 6-well plate (Falcon, 1.5 membranes per well) together with logarithmic phase CDI+ DL4577. After 5 min of incubation at 37° C. with shaking, DL4793 target cells (16 h culture) were added at a 20/1 inhibitor to target ratio. Mixed cultures were incubated at 37° C. with shaking (130 rpm) and viable counts were determined at the times indicated (filled bars). A control lacking added PET membrane is shown by open bars. Similar results were obtained with polycarbonate membranes. These results support the model that growth inhibition mediated by cdiAB requires cell-to-cell contact, designated as contact-dependent inhibition (CDI).

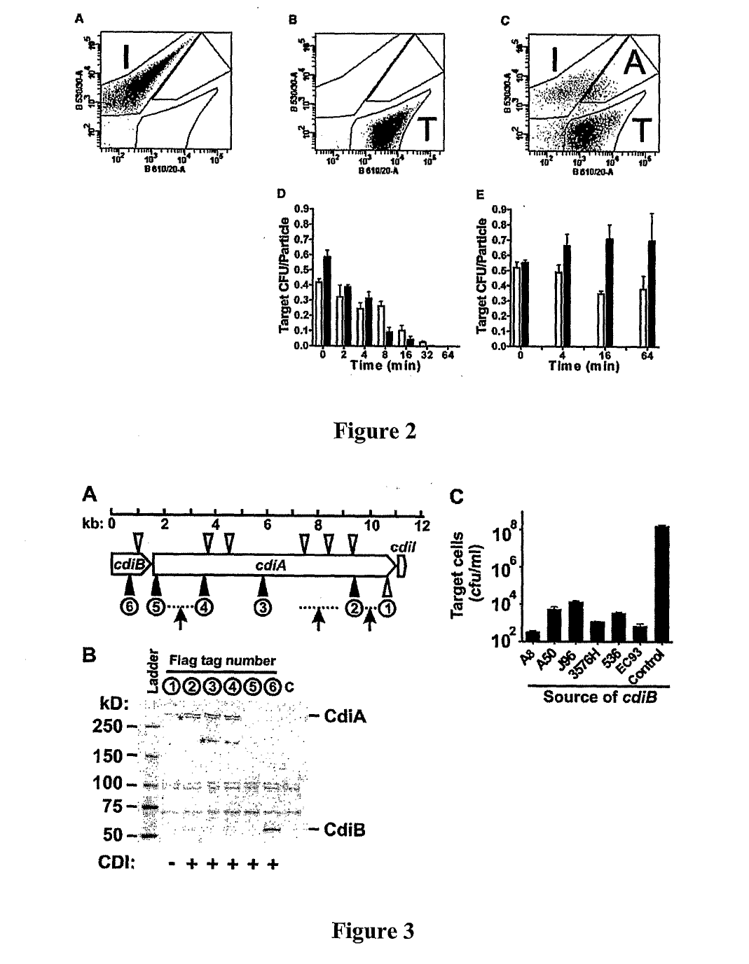

To address the possibility that the inhibitory factor could be an unstable secreted molecule that is only effectively delivered to target cells in close proximity, the inventors mixed fluorescently labeled cdiA+B+ inhibitory cells (GFP-labeled) with E. coli K-12 target cells [Discosoma red (DsRed)-labeled], and after increasing times of incubation with shaking, the cell mixtures were sorted by fluorescence-activated cell sorting (FACS). The inventors observed three cell populations, corresponding to green inhibitory cells, red target cells, and cell aggregates containing at least one inhibitory and one target cell per aggregate particle (FIG. 2A to 2C). Here, FACS and cell viability analyses of GFP- and DsRed-labeled E. coli were carried out as previously described. Panel A depicts results of CDI⁺ inhibitor cells “I” constitutively expressing GFP (DL4905). Panel B depicts results of E. coli target cells “T” constitutively expressing DsRed (D14920). Panel C depicts results from the experiment where the inhibitory and target cells described above were mixed at a 1:4 inhibitor-to-target ratio and analyzed after 16 min of growth with shaking at 37° C. The aggregated cell population “A” contained at least one inhibitor cell and one or more target cells per particle. The target population contained free target cells that were not associated with inhibitor cells.

The appearance of the aggregated particles was dependent upon cdiAB, indicating that CdiAB mediates intercellular binding. If cell-to-cell contact were required for growth inhibition, then the viability of target cells bound to inhibitory cells should decrease more rapidly than the viability of free target cells (FIG. 2D). Here, FACS sorting was used to isolate the free and inhibitor-bound target populations (in FIG. 2C). Time (min) Viability was scored as colony-forming units per particle sorted from each gated population. Free target cells are shown as open bars, and inhibitor-bound target cells are shown as solid bars. The viability of aggregated targets compared with free targets decreased at time points of 8 min and longer, indicating that observable growth inhibition occurred after 4 to 8 min of contact. Although the viability of free targets was only marginally reduced at times up to 8 min, at later times viability was significantly reduced, albeit at a lesser rate than for aggregated targets (FIG. 2D). It is likely that the observed reduction in viability of “free” target cells was primarily the result of prior contact with cdiA+B+ inhibitory cells and release from cell aggregates.

The rapid decrease in viability of the aggregated targets might be due to a nonspecific effect of intercellular binding. FACS analysis was carried out using a cdiA mutant (FIG. 3A, cdiA-FLAG1) that no longer conferred CDI but retained intercellular adhesion. More specifically, 15-bp linkers were inserted randomly into the cdiAB DNA region, generating either stop codons (top triangles) or 5 amino acid non-polar insertions (bottom triangles). FLAG tags were introduced within each of these nonpolar insertions. Solid triangles indicate insertions that did not affect CDI, whereas open triangles indicate insertions that disrupted CDI. Potential CdiA processing sites (arrows) were predicted from estimated protein sizes obtained (see 3B below). Under these conditions, the viability of aggregated target cells was not reduced compared to free target cells over the same time course (FIG. 2E: As in (2D), except that CDI⁻ E. coli containing cdiA-FLAG (D 14955) was mixed with target cells (DL4920)), showing that the rapid decrease in viability of target cells bound to inhibitory cells required CDI activity. These results strongly indicate that CdiAB mediates growth inhibition through cell-to-cell contact.

The inventors cloned a DNA region from E. coli EC93 that conferred a CDI+ phenotype to E. coli K-12 and generated 15 base-pair (bp) insertions within the region. Stop codon insertions within open reading frames (ORFs) designated cdiA and cdiB (GenBank accession no. DQ100454) abolished CDI activity (FIG. 3A), showing that the cdiA and cdiB ORFs were necessary for CDI. In addition, the inventors identified a small ORF (FIG. 3A, cdil) adjacent to cdiA that conferred full immunity to CDI (FIGS. 9A and 9B), explaining why cells expressing cdiAB do not inhibit their own growth. More specifically, as shown in panel A, the cdiI ORF from E. coli EC93 (FIG. 3A) was cloned in opposite orientations in plasmid pCCI to generate plasmid pDAL664 with the lac promoter in the same orientation as cdiI and plasmid pDAL664-REV in the reverse orientation. Plasmids were individually transformed into cdiABI-E. coli K-12 strain EPI300, and tested for immunity to CDI+ inhibitory cells (DL4577) as described in Materials and Methods. Panel B depicts the results obtained using EPI300(pDAL664) are depicted by black squares, EPI300(pDAL664-REV) are depicted by black circles and control E. coli EPI300 containing plasmid pCCI alone is also shown (triangles).

The translated cdiA and cdiB ORFs showed significant amino acid sequence identity with two-partner secretion proteins that are proteolytically processed during export to the cell surface. Using FLAG epitope tagging (FIG. 3A), the inventors found that CdiA was expressed as a 303-kD protein on the cell surface, which was then processed to 284-kD and 195-kD proteins (FIG. 3B). Here, E. coli containing CdiA and B proteins labeled with FLAG tags (lanes 1 to 6) or no FLAG tag control (lane C) were analyzed by SDS-PAGE and immunoblotting with monoclonal antiserum to FLAG. A molecular weight ladder and CdiA and CdiB protein positions are shown. Asterisks indicate CdiA fragments, and CDI phenotypes are shown at the bottom. A FLAG insertion in the cdiB ORF yielded a 56-kD protein, consistent with the predicted size of CdiB. Proteolytic fragments of CdiA were detected in the growth medium but were not growth-inhibitory (FIGS. 7A and 7B), indicating that the secreted forms of CdiA are inactive.

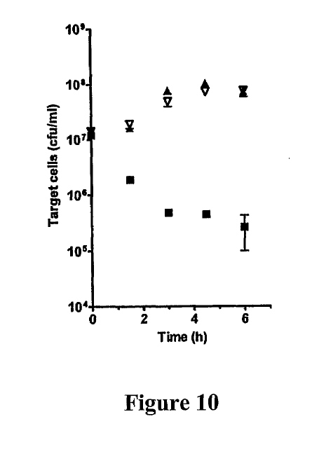

High amino acid sequence identity was found between CdiA/CdiB and predicted proteins from uropathogenic E. coli (UPEC), including strain 536. Complementation analysis indicated that UPEC 536 and four additional UPEC strains contain genes that are functional homologs of cdiB (FIG. 3C: cdiB homologs from five UPEC isolates and cdiB from EC93 were cloned in plasmid pCC1 and tested for CDI complementation in cdiA⁻B⁻ E. coli (DL4958). Vector control plasmid pCC1 is shown (control)) and cdiA (FIG. 10). Here, the cdiA homologue in UPEC 536 was cloned into plasmid pCCI under lac promoter control to generate plasmid pDAL665. Plasmid pDAL665 was then transformed into cdiA-I3+E. coli (EPI300 containing a stop codon mutation within cdiA) to test complementation. DL4793 target cell viability was determined in mixed cultures containing E. coli cdiA⁻B⁺ (pDAL665)(closed squares) and controls containing either E. coli cdiA⁻B⁻ (open triangle) or E. coli cdiA⁻B⁻(pDAL665) (closed triangle).

Bioinformatic analysis showed that Yersinia pestis (plague) and Burkholderia, pseudomallei (melioidosis) also encode possible CdiAB homologs. Filamentous hemagglutinin from Bordetella pertussis (whooping cough) appeared more distantly related, sharing sequence identity to CdiA primarily in the amino-terminal portion of the protein.

The cdiAB homologs in UPEC 536 are present within pathogenicity island II, but a cdiI homolog is not present, nor is it found in the sequenced genome of UPEC CFT073, which also contains a cdiAB homolog. This observation suggests that cdiAB expression in UPEC strains would inhibit their growth. Pathogenicity island II in UPEC 536 also contains a pyelonephritis-associated pili (pap) operon closely linked to cdiAB. Because Pap pili are expressed at the cell surface, the inventors tested the possibility that pili expression might affect CDI because contact between CdiA and the target cell surface could be blocked. E. coli K-12 constitutively expressing P pili or S pili showed resistance to CDI, whereas cells expressing type 1 pili were 10³ to 10⁴-fold more sensitive to growth inhibition. Thus, resistance to CDI conferred by P and S pili involves specific interaction(s) and is not likely to be the result of nonspecific steric hindrance that blocks cell-to-cell contact.

Many UPEC strains contain fim (type 1 pill), pap (P pill), and sfa (S pili) operons. The expression of these pili types is normally subject to reversible off/on switching, generating diversity within bacterial populations by a differentiation mechanism. Such a mechanism might play a role in the temporal control of the differentiation observed for UPEC strains inside bladder cells, during which the bacteria progress through distinct developmental stages, including a quiescent growth state. E. coli K-12 cells inhibited by CDI appear to be nonviable based on their lack of growth on agar medium and cellular metabolism may be significantly reduced. However, CDI-inhibited cells appeared to be viable based on exclusion of propidium iodide, a standard criterion for distinguishing viable and nonviable cells. The identification of this sophisticated mechanism in E. coli, with possible homologs in a broad range of species, opens the door for exploration of the potential roles of CDI in controlling bacterial development and pathogenesis.

### Strains, Plasmids, and Growth Conditions

E. coli strains and plasmids used in this study are commercially available or can be prepared following known procedures. Cells were grown on Luria-Bertani (LB) broth or agar supplemented with antibiotics at the following concentrations (unless otherwise noted): ampicillin (amp), 100 μg/ml; chloramphenicol (cam), 34 μg/ml; kanamycin (kan), 40 μg/ml; nalidixic acid (nal), 10 μg/ml; streptomycin (str), 100 μg/ml; tetracycline (tet), 12.5 μg/ml; rifampicin (rif), 150 μg/ml. Cultures were incubated at 37° C. in an environmental shaker apparatus (New Brunswick Series 25) at 225 rpm unless otherwise indicated.

### Competition Assay

E. coli EC93 and EPI100 CDI⁺ (DL4577) inhibitory cells were separately grown to logarithmic phase (OD₆₀₀ 0.35) in 50 ml LB medium containing appropriate antibiotics (500 ml baffled culture flask). EPI100 CDF (DL4527) cells were also included as a no inhibition control. MG1655 (DL4793) target cell cultures (16 h) were added at a 10 to 1 inhibitor to target cell ratio. Competitions were incubated at 37° C. with shaking at 225 rpm. Samples were taken hourly, ten-fold serially diluted in M9 salt solution (Sigma Co.) and 100 μl aliquots of serial dilutions were plated onto LB containing appropriate antibiotics for independent selection of inhibitor and target cells.

### Cosmid Library Construction and Nested Deletions

Cosmid library construction (pDAL660): A cosmid library of E. coli EC93 genomic DNA was made with an Epicentre pWEB::TNC Cosmid Cloning Kit. Briefly, DNA (−40 kb) was blunt-end cloned into the Smal site of pWEB::TNC, packaged using MaxPlax Lambda Packaging Extracts and transduced into EPI100 cells. Transductants were selected on LB-amp-cam agar and screened for growth inhibitory activity against MC4100 amp^{R} kan^{R}(DL159) target cells by incubating transductant and target cells together in LB containing appropriate antibiotics for 3 hours before sampling. Samples were ten-fold serially diluted in M9 salt solution and 5 μl aliquots were spotted onto LB-kan. CDI activity was scored by observation of the spotted target cell growth (single colonies versus confluent growth).

Nested deletions (pDAL660A): Nested deletions within pDAL660 were constructed with an Epicentre pWEB::TNC Deletion Cosmid Transposition Kit. Briefly, EZ::TN Transposase was used in vitro to generate unidirectional deletions, designated as pDAL660AX where “X” specifies a particular deletion. Deletion plasmids were transformed into EPI100 and transformants were selected for ampicillin-resistance and chloramphenicol-sensitivity. The positions of deletions were determined by sequencing using the pWEB::TNC sequencing primer. Deletion mutants were screened for growth inhibitory activity against EPI100 target cells (DL4527) by incubating transformant and target cells together in LB containing appropriate antibiotics for 3 hours before sampling. Samples were ten-fold serially diluted in M9 salt solution and 5 pl aliquots were spotted onto LB-cam. CDI activity was scored by observation of the spotted target cell growth (single colonies versus confluent growth).

### Linker Insertions

TnsABC* transposase (New England Biolabs) was used to randomly introduce single hops of a Tn7-based transprimer into deletion subclone pDAL66041-39. Transposon insertion sites were determined by DNA sequence analysis using oligonucleotide primers N and S (see GPS-LS Linker-Scanning System, New England Biolabs). To convert transposons to 15 bp linkers, each plasmid containing a transprimer insertion was digested with Pmel, ligated, and transformed into E. coli EPI100. This resulted in replacement of the transprimer sequence of each plasmid with a 15 bp insertion that either introduced an in-frame five amino acid sequence or an in-frame stop codon, designated as pDAL66041-39::TP⁻. Transformants were screened for growth inhibitory activity against MG1655 (DL4793) target cells by incubating transformant and target cells together in LB containing appropriate antibiotics for 3 hours before sampling. Samples were ten-fold serially diluted in M9 salt solution and 5 ul aliquots were spotted onto LB-tet. CDI activity was scored by observation of the spotted target cell growth (single colonies versus confluent growth).

### FLAG-Tag Linker Insertion (pDAL660A1-39::FLAG)

Plasmid pDAL66041-39::TP containing five amino acid insertions within CdiA (mutant numbers 1-64, 1-14, 1-67, 1-39, and 2-17) and CdiB (1-33) were linearized by digesting with PmeI. The appropriate FLAG-tag linker DNA sequence encoding DYKDDDDK was blunt-end cloned in-frame into the Pmel site and electroporated into TransforMax EPI100 (Epicentre). Amp^{R} colonies were screened for correct FLAG orientation by PCR, and assayed for growth inhibitory activity and immunity as described above.

### Screens and Quantitative Assays for Growth Inhibition and Immunity

A screen to identify bacteria with contact-dependent growth inhibitory activity (CDI) was developed to test cosmids, cosmid subclones, and transposon insertions as follows. Bacteria to be screened were grown to logarithmic phase (0D₆₀₀ 0.8) in 5 ml LB medium containing appropriate antibiotics. Overnight cultures (16 h) of target cells were added for an inhibitor to target cell ratio of 20 to 1. Competitions were then incubated for 3 h, ten-fold serially diluted in M9 salt solution, and aliquots (5 μl) of each dilution were spotted onto LB agar containing appropriate antibiotics for selection of target cells. CDI activity was scored by observation of the spotted target cell growth (single colonies versus confluent growth). EC93 or DL4577 was used as a positive control for inhibitory activity and EPI100 was used as a negative control for inhibitory activity in each experiment. A quantitative assessment of growth inhibition was carried out as above except that 100 μl aliquots of serial dilutions were plated on LB containing appropriate antibiotics to obtain viable colony counts of target cells.

A screen to detect bacteria immune to CDI-dependent growth inhibition was carried out by inoculating overnight cultures (16 h) of strains to be screened into LB cultures of logarithmic phase DH5a CDT⁺ (DL4608) inhibitory cells for an inhibitor to target ratio of 20 to 1. After 3 h of incubation, cultures were ten-fold serially diluted and spotted as above for the growth inhibition assay onto LB-str agar for selection of strains being tested. E. coli EC93 str^{R} was used as a positive control and EPI100 was used as a negative control for immunity to CDI. Quantitation of immunity was done as above for immunity screening except that 100 μl aliquots were plated on LB-str agar for viable colony counts of the tested strains.

### Growth Phase Assay

E. coli CDI⁺ EC93 inhibitor cells and MC4100 Nal^{R} (DL430) target cells were separately incubated in LB medium and grown to stationary phase (16 h) or logarithmic phase (0D₆₀₀=0.35). Inhibitor cells were then mixed with target cells and incubated at 37° C. with shaking (225 rpm). Stationary phase inhibitor cells were incubated with target cells at an inhibitor to target cell ratio of 500 to 1. Logarithmic phase inhibitor cells were incubated with target cells at an inhibitor to target cell ratio of 5 to 1. Samples were taken hourly, ten-fold serially diluted in M9 salt solution and 100 μl aliquots were plated onto LB containing appropriate antibiotics for independent selection of inhibitor and target cells. E. coli MC4100 (K-12) CDI⁻ inhibitor control cells were also included as a negative control for growth inhibitory activity.

### Chloramphenicaol Pretreatment Assay

E. coli EPI100 CDI⁺ inhibitor (DL4577) and EPI100 CDI− inhibitor control (DL4956) cells were separately inoculated into 50 ml of LB-amp medium in a 500 ml baffled culture flask and grown to logarithmic phase (OD₆₀₀˜0.3). Chloramphenicol was then added to a final concentration of 34 μg/ml and cultures were incubated for two additional hours. Target E. coli DL4793 culture (16 h) was added to chloramphenicol pretreated cells at an inhibitor to target ratio of 10 to 1. Samples were taken hourly, ten-fold serially diluted in M9 salt solution and 100 μl aliquots were plated onto LB containing appropriate antibiotics to obtain viable colony counts of inhibitor and target cells. Untreated inhibitor and inhibitor control cultures were included as a negative control for protein synthesis inhibition.

### Analysis of CDI at Different Inhibitor to Target Cell Ratios

Logarithmic growth phase E. coli DL4577 (0D₆₀₀˜0.35, 15 mL) were added to 125 mL flasks and inoculated with target E. coli DL4793 at different inhibitor to target cell ratios. Cells were incubated at 37° C. with shaking (150 rpm) and samples were obtained at 0, 5, 20, 60, 75, 90, 120, 135, 150, and 180 minutes. Samples were ten-fold serially diluted in M9 salt solution and plated on LB containing appropriate antibiotics for independent selection of inhibitor and target cells. CDI E. coli DL4527 was used as a negative control for growth inhibitory activity.

### Growth Inhibitory Activity Secretion Assays

E. coli EPI100 CDI⁺ inhibitor (DL4577) and EPI100 CDI⁻ control cells were separately grown to logarithmic phase (0D₆₀₀=0.35) in LB-amp medium and 16 h target MG1655 (DL4793) cells were added at a 10 to 1 inhibitor to target cell ratio. Samples were taken hourly, ten-fold serially diluted in M9 salt solution and 1001×1 aliquots were plated onto LB containing appropriate antibiotics to obtain viable counts of inhibitor and target cells. After one hour of co-incubation, a 15 ml sample from each culture was centrifuged at 6000×g for 1 min and supernatant solutions were filtered through a low protein binding 0.45 μm HT Tuffryn membrane (Pall Life Sciences) to remove any remaining cells. Target E. coli DL4793 cells (16 h culture) were added 1/200 (v/v) into 12.5 ml of filtered cell culture supernatant. Samples were taken hourly and viable target cell counts were determined as described above.

### Contact-Dependent Inhibitory Activity Assay

Polyethylene terephthalate (PET) track-etched membrane inserts (23 mm) of 0.4 μm or 8 μm pore size (Falcon) were placed in 6-well plates to create upper and lower culture wells. LB-amp medium (15 ml in a 125 ml flask) was inoculated with an overnight inhibitor culture of EPI100 CDI+ (DL4577), grown to logarithmic phase (0D₆₀₀˜0.35), and 2.5 ml was added to the top chambers. 3.2 ml of a 16 h culture of target MG1655 (DL4793) cells was added to the bottom chambers at a 20 to 1 ratio of inhibitor to target cells (estimated by OD₆₀₀). Cells were incubated at 37° C. with shaking (130 rpm) and sampled at 0, 1, 3, and 5 hours. Samples were ten-fold serially diluted in M9 salt solution and 100 p. 1 aliquots were plated onto LB containing appropriate antibiotics for independent selection of inhibitor and target cells. E. coli EPI100 CDI⁻ (DL4527) was used in place of DL4577 as a negative control for inhibitory activity.

### Pet Membrane Control

Several PET membranes (Falcon) were minced aseptically with dissecting scissors. PET membrane fragments (1.5 membranes/well) together with logarithmic phase CDI+ inhibitory strain DL4577 were added to the top chamber of 6-well plates containing a 0.4 μm membrane insert. Membranes were incubated with inhibitor cells for 5 minutes at 37° C. with shaking before adding DL4793 target cells (16 hour culture) at a 20 to 1 ratio of inhibitor to target (estimated by OD₆₀₀). Cells were incubated at 37° C. with shaking (130 rpm) and samples were obtained at 0, 1, 3, and 5 hours. Samples were ten-fold serially diluted in M9 salt solution and plated onto LB containing appropriate antibiotics for independent selection of inhibitor and target cells. Wells containing DL4577 and DL4793 without membrane fragments were used as a positive control of target cell inhibition.

### FACS Analysis

Flow cytometry was carried out using a BD Biosciences FACSAria instrument with a 10011 m sorting nozzle at low pressure. GFP-mut3 and DsRed were excited using a 488 nm blue laser and detected using 530/30 nm and 610/20 nm filters respectively. DsRed labeled target E. coli were prepared by overnight growth of DL4920 in TB-amp broth with 0.8 mM IPTG to induce expression of DsRed from pDAL672. Overnight cultures of both target and inhibitor E. Coli were separately diluted 100-fold into fresh TB-amp broth with 0.8 mM IPTG and grown to early log phase (0D₆₀₀=0.2) prior to mixing. Mixed cultures (6 ml) were incubated at 37° C. in 50 ml baffled culture flasks with shaking at 250 rpm for the times indicated. Inhibitor-bound target cells and free target cells were isolated by sorting 10,000 particles of each cell population into separate tubes containing phosphate buffered saline (1 ml). Samples were then vortexed vigorously (45 s) to disrupt intercellular aggregates and plated in quadruplicate (0.1 ml per plate) on LB-tet (5 μg/ml) agar plates. Target cell viability was determined by counting colony-forming units per particle sorted from each gated population.

### Immunoblot Analysis

EPI100 pDAL66041-39::FLAG strains and DL4577 (no FLAG, negative control) were grown in LB medium to an OD₆₀₀=0.8, and 150 gl of culture was centrifuged at 16,000×g for 30 s. Pellets were resuspended in 30 μl LDS sample buffer with reducing agent (Invitrogen). Samples were incubated at 75° C. for 15 minutes and loaded onto a 12-well NuPAGE Novex 3-8% Tris-acetate gel (Invitrogen) along with 1 μl Odyssey protein molecular weight marker (LI-COR) and electrophoresis was performed according to manufacturer (Invitrogen). Proteins were transferred to nitrocellulose in Tris-glycine transfer buffer using the XCell H Blot Module (Invitrogen) as directed by manufacturer. The membrane was rinsed with phosphate-buffered saline (PBS) and blocked overnight at 4° C. in Odyssey blocking buffer:PBS (1:1). Mouse anti-FLAG M2 antibody (Sigma) 1:500 in Odyssey blocking buffer:PBS was added to the membrane and incubated for 1 h at room temperature. Blots were rinsed in PBS+0.05% Tween-20 prior to incubation with goat anti-mouse IgG Alexa Fluor 680-allophycocyanin (Molecular Probes) 1:2500 in Odyssey blocking buffer:PBS+0.1% Tween-20 for 1 h at 23° C. The blot was washed in PBS+0.1% Tween-20, then PBS, and air-dried overnight. Blots were imaged on the 700 channel of the Odyssey Infrared Imaging System (LI-COR).

### Complementation Analysis using cdiA and cdiB Genes from UPEC Strains cdiB

complementation: Homologues of cdiB from uropathogens A8, A50, J96, 3576H, and 536 were amplified by polymerase chain reaction (PCR) using selected oligonucleotide primers based on the cdiB DNA sequence from EC93. The 1.8 kb PCR products were blunt-end cloned into the Eco721 site of plasmid vector pCC 1 under lac promoter control using the Copy-Control PCR Cloning Kit (Epicentre) according to manufacturer instructions. Briefly, PCR products were precipitated, end-filled and ligated into the Eco721 site of pCC1. Ligations were electroporated into TransforMax EPI300 (Epicentre) and chloramphenicol-resistant colonies were screened for insert orientation. Complementation was carried out by transforming cloned cdiB genes into E. coli EPI300 pDAL660A1-39::2-7Tr (DL4958), which contains a non-polar 5 amino acid insertion within the cdiB gene and lacks growth inhibitory activity but maintains immunity. DL4958 transformed with EC93 cdiB (pDAL662) was used as a positive control for complementation and DL4958 containing plasmid pCC I was used as a negative control for complementation. Strains to be tested were grown to logarithmic phase (0D₆₀₀˜0.35) in 12.5 ml LB containing appropriate antibiotics in the presence of CopyControl induction solution (Epicentre) for maximal CdiB expression. 16 h target cell cultures (MG1655, DL4793) were added at an inhibitor to target cell ratio of 10 to 1. Competitions were incubated at 37° C. with shaking at 225 rpm and sampled hourly. Samples were ten-fold serially diluted in M9 salt solution and 100 ill aliquots were plated onto LB containing appropriate antibiotics for independent selection of inhibitor and target cells.

cdiA complementation: For complementation with cdiA, an 11 kb DNA fragment from UPEC 536 encompassing cdiA was amplified using PCR and oligonucleotide primers 875 and 877. The fragment was blunt-end cloned into the Eco721 site of pCC 1 as for cdiB above to generate plasmid pDAL665. Complementation was carried out by transforming the plasmid into E. coli EPI300 containing pDAL660A1-39::1-32TP⁻ (DL4957), which contains a stop codon within the cdiA gene and lacks growth inhibitory activity but maintains immunity. Complementation was tested as for cdiB above.

### Cloning and Analysis of cdiI

A 401 bp DNA fragment adjacent to cdiA was amplified from E. coli EC93 by PCR using oligonucleotides 883 and 884. The DNA fragment was blunt-end cloned into vector pCCI as described earlier, electroporated into EPI300, and chloramphenicol-resistant colonies were screened for insert orientation. Plasmids pDAL664 and pDAL664-REV were generated containing a lac promoter controlling the putative ORF designated cdil in the same transcriptional orientation or reverse orientation, respectively. Analysis of immunity was performed by growing CDI+ inhibitor cells (DL4577) to logarithmic phase (0D₆₀₀ 0.35) and adding transformant target cells [16 h growth in LB-cam medium with CopyControl induction solution (Epicentre) to maximize cdil expression] at an inhibitor to target ratio of 20 to 1. Mixed cultures were incubated at 37° C. with shaking (225 rpm) and sampled hourly. Samples were ten-fold serially diluted in M9 salt solution and plated on LB containing appropriate antibiotics for viable counts of inhibitor and target cells.

### Pili Expression and Resistance to Inhibition

E. coli FEB101 rif^{R} target cells (pill) were transformed with plasmids expressing P pili (pDAL230B), S pili (pANN801-13), type 1 pili (pSH2), or plasmid vector alone (pBR325). Target cells (16 h) were incubated with logarithmic phase (0D600 0.35) CDI⁺ inhibitory E. coli (DL4577), and samples were taken three hours after incubation, serially diluted in M9 salt solution and plated onto LB containing appropriate antibiotics for viable counts of inhibitor or target cells. No inhibition of target cell growth was observed using cDr E coli DL4527 in place of CDI⁺ DL4577 inhibitory cells, showing that the growth inhibition observed is CDI-dependent.

Thus, specific embodiments and applications of bacterial contact inhibition and methods therefore have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

1. A test system for investigation of bacterial contact inhibition comprising an isolated nucleic acid sequence encoding a peptide of a bacterial contact inhibition system, wherein the sequence is selected from the group consisting of SEQ. ID. No. 1, SEQ. ID. No. 2, and SEQ. ID. No. 3, or a homolog thereof having at least 70% identity with SEQ. ID. No. 1, SEQ. ID. No. 2, and SEQ. ID. No. 3, respectively.

2. The isolated nucleic acid sequence of claim 1 wherein the sequence is SEQ. ID. No. 1 or a homolog thereof having at least 70% identity.

3. The isolated nucleic acid sequence of claim 1 wherein the sequence is SEQ. ID. No. 2 or a homolog thereof having at least 70% identity.

4. The isolated nucleic acid sequence of claim 1 wherein the sequence is SEQ. ID. No. 3 or a homolog thereof having at least 70% identity.

5. A recombinant vector comprising a sequence selected from the group consisting of SEQ. ID. No. 1, SEQ. ID. No. 2, and SEQ. ID. No. 3, or a homolog thereof having at least 70% identity with SEQ. ID. No. 1, SEQ. ID. No. 2, and SEQ. ID. No. 3, respectively, and an information that the sequence is functionally associated with bacterial contact inhibition.

6. A cell comprising the vector of claim 5, and an information that the cell expressed a sequence that is functionally associated with bacterial contact inhibition.

7. The cell of claim 6, wherein the cell is a bacterial cell.

8. An isolated polypeptide of a bacterial contact inhibition system having a sequence selected from the group consisting of SEQ. ID. No. 4, SEQ. ID. No. 5, and SEQ. ID. No. 6, or a homolog thereof having at least 80% identity with SEQ. ID. No. 4, SEQ. ID. No. 5, and SEQ. ID. No. 6, respectively.

9. The isolated polypeptide of claim 8 having a sequence of SEQ. ID. No. 4 or a homolog thereof having at least 80% identity.

10. The isolated polypeptide of claim 8 having a sequence of SEQ. ID. No. 5 or a homolog thereof having at least 80% identity.

11. The isolated polypeptide of claim 8 having a sequence of SEQ. ID. No. 6 or a homolog thereof having at least 80% identity.

12. A method of reducing bacterial contact inhibition comprising a step of contacting the bacterium with a compound that binds to at least one component of the bacterial contact inhibition system at a concentration effective to reduce bacterial contact inhibition.

13. The method of claim 12 wherein the compound is an antibody or fragment thereof.

14. The method of claim 13 wherein the antibody is an isolated monoclonal antibody.

15. The method of claim 13 wherein the antibody binds to a protein having a sequence of SEQ. ID. No. 4 or SEQ. ID. No. 5.

16. A method of treating a chronic bacterial infection comprising:
ascertaining that a bacterium expresses a nucleic acid that encodes for a component of a bacterial contact inhibition system; and
providing instructions to contact the bacterium with a compound that binds to at least one component of the bacterial contact inhibition system at a concentration effective to reduce bacterial contact inhibition; and
optionally providing instructions to contact the bacterium with an antibiotic drug.

17. The method of claim 16 wherein the bacterium is selected from the group consisting of Escherichia coli, Yersinia pestis, and Burkholderia pseudomallei.

18. The method of claim 16 wherein the compound is an antibody or a fragment thereof.

19. The method of claim 16 wherein the antibiotic drug is selected from the group of a beta-lactam antibiotic, a cephalosporin antibiotic, a tetracylin antibiotic, a macrolide antibiotic, and a fluoroquinoline antibiotic.
